(19) United States
(12) Patent Application Publication
(10) Pub. No.: US 2022/0202461 A1
(43) Pub. Date: Jun. 30, 2022

(54) ASSEMBLY FOR POSITIONING AND POSITIONALLY IMMOBILIZING AN INFLATABLE BALLOON WITHIN A VERTEBRAL BODY

(71) Applicant: Safe Orthopaedics, Eragny-Sur-Oise (FR)

(72) Inventors: Dominique Petit, Verton (FR); Steven Van Gaalen, Utrecht (NL)

(21) Appl. No.: 17/601,810

(22) PCT Filed: Apr. 9, 2020
(86) PCT No.: PCT/EP2020/060145

§ 371 Date: Oct. 6, 2021

(30) Foreign Application Priority Data
Apr. 9, 2019 (FR) FR1903788

(51) Int. Cl. A61B 17/88; A61B 17/16; A61B 17/90; A61F 2/44

(52) U.S. Cl. CPC A61B 17/8855; A61F 2/441; A61B 17/90; A61B 17/1671

(57) ABSTRACT

The invention relates to an assembly (1) for positioning and positionally immobilizing an inflatable balloon (3) within a vertebral body, comprising an access piece (2) providing access to the vertebral body, which has a cannula and is of elongate shape, and an inflatable balloon (3) mounted at the end of a catheter (4) able to be connected to a fluid-delivery unit, the assembly comprising balloon (3) and catheter being designed to pass slidingly through the access piece (2), characterized in that the access piece (2) comprises means of anchorage in the vertebral body, the assembly further comprising means for positionally immobilizing the catheter provided with the balloon (3) on the access piece (2).

### TECHNICAL FIELD OF THE INVENTION

This invention relates to the field of vertebral stabilization, and more particularly to the restoration of height (reduction) of a collapsed vertebra.

The invention relates more particularly to an assembly for positioning and positionally immobilizing an inflatable balloon within a vertebral body, comprising an access piece providing access to the vertebral body, which has a cannula and is of elongate shape, and an inflatable balloon mounted at the end of the catheter able to be connected to a fluid-delivery unit, the balloon and catheter assembly being designed to pass slidingly through the access piece.

“Catheter” in the present application refers to a thin and flexible tube allowing for the injection and/or evacuation of a liquid.

### PRIOR ART

Kyphoplasty is one of the techniques used to reduce a collapsed vertebra. With this technique, the vertebral height is restored by means of a balloon, or any other device that can be expanded within the vertebral body, which is placed in the vertebral body prior to cement injection. The balloon, or more generally the device, is inserted percutaneously through the pedicle. Using a catheter, syringe, or similar device at the end of which it is mounted, the balloon is guided through a working cannula into the vertebral body and inflated to reduce the fracture. After the fracture has been reduced, the balloon is deflated and removed. The resulting cavity is then filled with bone cement to stabilize the fracture.

One of the problems encountered with this technique is the positioning of the balloon within the vertebra to be treated. If the balloon is positioned too far backward or too far forward in the vertebra, this will affect the straightening of the vertebra, as shown in FIG. 10. And, as shown in FIG. 10b, a balloon 3 that is not sufficiently advanced within the vertebra 100 does not fully restore the height of the vertebra: a mismatch between the height of the straightened vertebra and the optimal straightening height is visible. On the contrary, as illustrated in FIG. 10c, the positioning of a balloon 3 further into the vertebral body allows for an optimal straightening of the vertebra 100.

Another problem encountered with this technique is keeping the balloon within the vertebra to be straightened. Indeed, as a result of the forces exerted on the balloon by the vertebra during its straightening, the balloon will tend to slide towards the area in which the least stress is exerted, so that the effect of the balloon on the restoration of the vertebra's height may be reduced. The less the balloon is advanced into the vertebral body, the less the height recovery of the vertebra will be.

This invention is designed to remedy these problems by providing an assembly that stabilizes the positioning of the inflatable balloon within the vertebral body, thereby ensuring optimal straightening of the collapsed vertebra, regardless of the degree of compression of the latter.

### OBJECT OF THE INVENTION

To this end, and according to a first characteristic, the invention provides an assembly for positioning and positionally immobilizing an inflatable balloon within a vertebral body, comprising an access piece providing access to the vertebral body, which has a cannula and is of elongated shape, and an inflatable balloon mounted at the end of a catheter able to be connected to a fluid delivery unit, the balloon and rod being designed to pass slidingly through the access piece, the assembly being remarkable in that the access piece comprises means for anchorage in the vertebral body and in that it further comprises means for positionally immobilizing the catheter provided with the balloon on the access piece.

The access piece, due to its means for anchorage in the vertebral body, forms, once anchored in the vertebral body, a stabilized channel in relation to the vertebra, while the immobilizing means prevent any sliding movement of the catheter through the access piece. The presence of the immobilizing means combined with the anchoring of the access piece thus allows the anchored access piece to establish a fixed point with respect to the vertebra but also with respect to the catheter. This facilitates balloon placement and ensures that the balloon remains in position within the vertebra. The positioning of the balloon is therefore no longer random, as is the case with current kyphoplasty systems, as any slippage of the balloon during inflation is avoided.

Advantageously, the means of anchorage consists of at least one mesh net.

Advantageously, the access piece is an implant.

According to an embodiment, the access piece comprises an extended implant instrument.

Advantageously, the implant is an anchor pin.

According to another variant, the access piece is a cannula. Advantageously, the cannula is designed to have a slidingly mounted drilling instrument extending through the cannula, said drilling instrument has a shaft with a tapered distal end to permit drilling of the vertebral body, the cannula and the drilling shaft are sized in relation to each other so that the tapered distal end of the drill rod extends beyond the cannula.

Advantageously, the immobilizing means are arranged to allow for adjustment of the positioning of the catheter in relation to the access piece and to immobilize the catheter in the fixed position with respect to said piece.

Advantageously, the immobilizing means comprise a sleeve slidingly mounted on the catheter and a means for securing the sleeve to the access piece.

Advantageously, the sleeve comprises a longitudinal mounting slot for the passage of the catheter through said sleeve, said slot being arranged to hold the sleeve on the catheter while allowing it to slide along the latter.

### BRIEF DESCRIPTION OF DRAWINGS

Further characteristics and embodiments of the invention will be apparent from the detailed description of the invention that follows with reference to the attached figures, in which:

FIG. 1 shows an assembly for positioning and positionally immobilizing an inflatable balloon within a vertebral body according to a first embodiment of the invention;

FIG. 2 shows the assembly of FIG. 1 in position within a vertebra, with the balloon shown in position in the vertebral body;

FIG. 3 shows a detailed view of the proximal part of the assembly from FIG. 1 ensuring the immobilization of the balloon;

FIG. 4 shows a detailed view of the distal part of the assembly from FIG. 1 ensuring the immobilization of the balloon;

FIG. 5 shows an assembly for positioning and positionally immobilizing an inflatable balloon in position within a vertebral body according to a second embodiment;

FIG. 6 shows the assembly of FIG. 5 in position within a vertebra, with the balloon shown in position within the vertebral body;

FIG. 7 shows a detailed view of the proximal part of the assembly from FIG. 5 ensuring the immobilization of the balloon;

FIG. 8 shows a detailed view of the distal part of the assembly from FIG. 5 ensuring the immobilization of the balloon;

FIG. 9 shows a detailed view of the balloon immobilization;

FIG. 10 shows the impact of balloon positioning on vertebral straightening within a vertebra.

For the sake of clarity, identical or similar elements from the various embodiments are marked with identical reference signs throughout the figures.

### DETAILED DESCRIPTION OF INVENTION

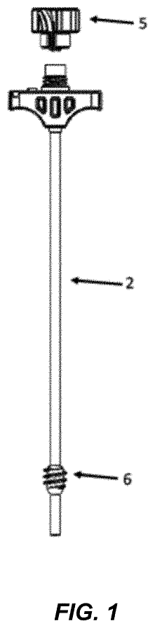

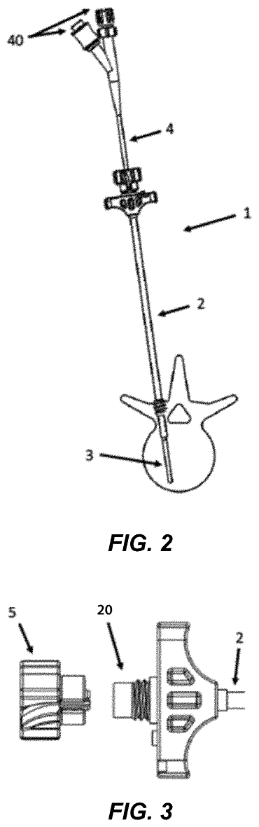

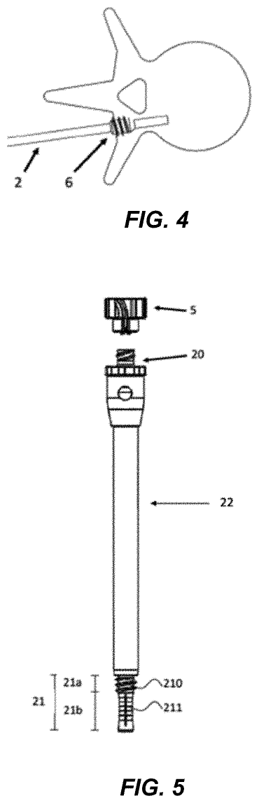

In relation to FIGS. 1 to 4, an embodiment is described consisting of an assembly 1 allowing for the positioning and positional immobilization of an inflatable balloon 3 within a vertebral body.

The assembly 1 comprises two sub-assemblies: a first sub-assembly comprising an access piece in the vertebral body, said piece being elongated and cannulated, and a second sub-assembly formed by an inflatable balloon 3 mounted at the end of an advantageously flexible catheter 4. The balloon/rod (catheter) sub-assembly is slidingly arranged through the access piece 2. In the illustrated embodiment, the access piece is a rigid cannula. Advantageously, the catheter 4 comprises a means for connection 40 to a fluid delivery unit.

According to the invention, the assembly 1 comprises means for stabilizing the balloon 3 with respect to the vertebra. The stabilizing means of the balloon 3 comprise first means for ensuring the positional immobilization of the catheter, carrying the balloon 3 at its end, on the access piece 2 and second means for ensuring anchorage of the access piece 2 in the vertebral body.

Advantageously, the immobilizing means are in the form of a sleeve 5 slidingly mounted along the catheter 4.

According to a particular embodiment illustrated in FIG. 3, the sleeve 5 and the access piece 2 comprise mutual coupling means. In the embodiment illustrated in FIG. 3, the sleeve 5 is screwed onto a hooking end 20 provided at the proximal end of the access piece 2.

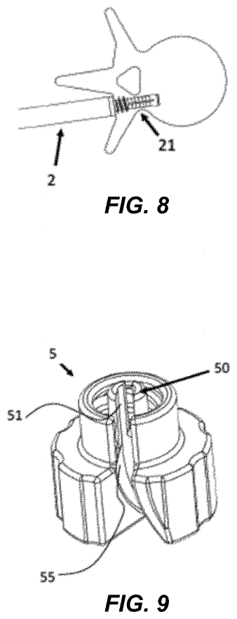

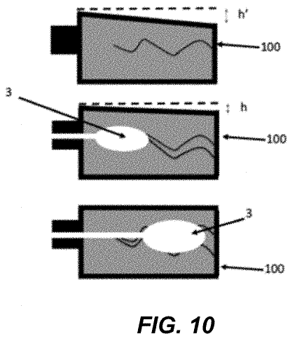

In the described embodiment, the sleeve 5 has a longitudinal mounting slot 55 for passage of the catheter 4 (FIG. 9). The mounting slot 55 is designed to hold the sleeve 5 on the catheter while allowing it to slide along the catheter.

The sleeve 5 also includes a retaining end 50 for the catheter 4 when it is in place on the access piece 2. The retaining end 50 is a bendable end of the clamp type. It has a side wall provided with a passage slot extending along its entire length and communicating with the mounting slot 55. The retaining end 50 is arranged to fit inside the hooking end 20 of the access piece 2. The retaining end 50 has a truncated cone-shaped internal wall sized so that, when it is inserted into the hooking end 20 of the access piece 2, its cross-section narrows when in contact with the internal wall of the hooking end 20 so as to grip and immobilize the balloon 3 catheter 4.

The means for anchorage consists, in the illustrated embodiment, of a mesh net 6 provided on the peripheral wall of the cannulated piece 2.

Advantageously, the cannula 2 is configured to have a drilling instrument slidingly mounted therethrough and having a shaft with a tapered distal end for drilling the vertebral body. The cannula and the drilling shaft are designed so that the tapered distal end of the drilling shaft extends beyond the cannula. The cannula and the drilling instrument thus advantageously comprise a Jamshidi™ type instrument.

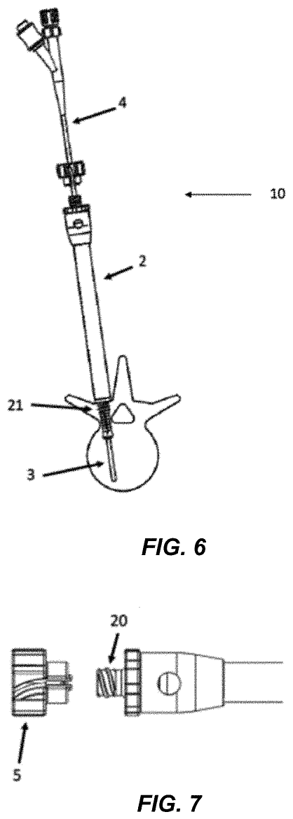

FIGS. 5 to 8 illustrate another embodiment of an assembly 10 for positioning and positionally immobilizing an inflatable balloon 3. In this embodiment, the assembly 10 for positioning and positionally immobilizing the balloon has all the characteristics of the previously described assembly, and in particular the part for immobilizing the balloon 3 with respect to the access piece. It differs, however, in the construction of the access piece 2 and the means for anchorage used. According to this embodiment, the access piece 2 is not composed of a cannula as in the previously described embodiment, but of an instrument coupled to an implant. More particularly, and as illustrated in FIG. 5, the access piece 2 comprises a tube 22 provided at its proximal end with a cannulated implant 21. In the illustrated embodiment, the implant 21 is an anchor plug.

The anchor plug 21, in the illustrated embodiment, is in the form of a cannulated cylindrical plug body comprising two parts: an upper part 21a provided with an external helical thread 210 and an expandable lower part 21b. The plug 21 may be made of a thermoplastic material, preferably PEEK, or of metal. The upper portion 210 provided with the thread allows primary anchoring of the plug in the vertebral body. It defines a zone extending approximately over one third of the length of the plug 4. The lower part 21b has a longitudinal slot delimiting the arms that can expand radially. Advantageously, the longitudinal slots are not open at the proximal end of the plug. The arms are thus attached to each other at the proximal end. The lower part 21b also includes external radial notches 211 arranged to block the movement of the plug in a direction opposite to that of its insertion in the vertebral body, and thus preventing any untimely withdrawal of the plug when the latter is anchored in the vertebral body.

As is well understood, the plug 21 constitutes the means of anchorage to the vertebral body.

The invention is described in the foregoing using embodiments. It is understood that a person skilled in the art would be able to carry out different variants of the invention without going beyond the scope of the invention.

1. Assembly (1, 10) for positioning and positionally immobilizing an inflatable balloon (3) within a vertebral body, comprising an access piece (2) providing access to the vertebral body, which has a cannula and is of elongate shape, and an inflated balloon (3) mounted at the end of a catheter (4) able to be connected to a fluid-delivery unit, the assembly comprising balloon (3) and catheter being designed to pass slidingly through the access piece (2), characterized in that the access piece (2) comprises means of anchorage in the vertebral body, the assembly further comprising means for positionally immobilizing the catheter provided with the balloon (3) on the access piece (2).

2. Assembly (1, 10) for positioning and positionally immobilizing an inflatable balloon (3) according to claim 1, characterized in that the means of anchorage consists of at least one mesh net (6).

3. Assembly (1, 10) for positioning and positionally immobilizing an inflatable balloon (3) according to claim 1 or claim 2, characterized in that the access piece (2) is an implant.

4. Assembly (1, 10) for positioning and positionally immobilizing an inflatable balloon (3) according to claim 1 or claim 2, characterized in that the access piece (2) comprises an extended instrument with an implant.

5. Assembly (1, 10) for positioning and positionally immobilizing an inflatable balloon (3) according to claim 3 or claim 4, characterized in that the implant is an anchor plug (21).

6. Assembly (1, 10) for positioning and positionally immobilizing an inflatable balloon (3) according to claim 1 or claim 2, characterized in that the access piece (2) is a cannula.

7. Assembly (1, 10) for positioning and positionally immobilizing an inflatable balloon (3) according to claim 6, characterized in that the cannula is configured to have a slidingly mounted drilling instrument extending therethrough, said drilling instrument having a shaft with a tapered distal end so as to permit drilling of the vertebral body, the cannula and the drilling shaft being sized relative to each other so that the tapered distal end of the drilling shaft extends beyond the cannula.

8. Assembly (1, 10) for positioning and positionally immobilizing an inflatable balloon (3) according to any one of the preceding claims, characterized in that the immobilizing means are arranged to allow for adjustment of the positioning of the catheter in relation to the access piece (2) and to immobilize the catheter in the selected position in relation to said piece.

9. Assembly (1, 10) for positioning and positionally immobilizing an inflatable balloon (3) according to any one of the preceding claims, characterized in that the immobilizing means comprise a sleeve (5) slidingly mounted on the catheter (4) and means for securing the sleeve to the access piece (2).

10. Assembly (1, 10) for positioning and positionally immobilizing an inflatable balloon (3) according to claim 9, characterized in that the sleeve (5) comprises a longitudinal mounting slot (55) to pass the catheter through said sleeve, said mounting slot (55) being designed to hold the sleeve (5) onto the catheter while allowing it to slide along the latter.
